(19) United States
(12) Patent Application Publication
(10) Pub. No.: US 2008/0306582 A1
(43) Pub. Date: Dec. 11, 2008

(54) Implantable medical devices with elastomeric copolymer coatings

(76) Inventors: Yunbing Wang, Synnyvale, CA (US); Bin Huang, Pleasanton, CA (US)

Correspondence Address: SQUIRE, SANDERS & DEMPSEY LLP, 1 MARITIME PLAZA, SUITE 300, SAN FRANCISCO, CA, 94111, US

(21) Appl. No.: 11/810,464

(22) Filed: Jun. 5, 2007

(51) Int. Cl. A61F 2/06; C08G 63/08

(52) U.S. Cl. 623/1.15; 528/354

(57) ABSTRACT

Implantable medical devices with elastomeric copolymer coatings are disclosed.

### BACKGROUND OF THE INVENTION

1. Field of the Invention

This invention relates to elastomeric coatings for implantable medical devices.

2. Description of the State of the Art

This invention relates to radially expandable endoprostheses, which are adapted to be implanted in a bodily lumen. An “endoprosthesis” corresponds to an artificial device that is placed inside the body. A “lumen” refers to a cavity of a tubular organ such as a blood vessel.

A stent is an example of such an endoprosthesis. Stents are generally cylindrically shaped devices, which function to hold open and sometimes expand a segment of a blood vessel or other anatomical lumen such as urinary tracts and bile ducts. Stents are often used in the treatment of atherosclerotic stenosis in blood vessels. “Stenosis” refers to a narrowing or constriction of the diameter of a bodily passage or orifice. In such treatments, stents reinforce body vessels and prevent restenosis following angioplasty in the vascular system. “Restenosis” refers to the reoccurrence of stenosis in a blood vessel or heart valve after it has been treated (as by balloon angioplasty, stenting, or valvuloplasty) with apparent success.

The treatment of a diseased site or lesion with a stent involves both delivery and deployment of the stent. “Delivery” refers to introducing and transporting the stent through a bodily lumen to a region, such as a lesion, in a vessel that requires treatment. “Deployment” corresponds to the expanding of the stent within the lumen at the treatment region. Delivery and deployment of a stent are accomplished by positioning the stent about one end of a catheter, inserting the end of the catheter through the skin into a bodily lumen, advancing the catheter in the bodily lumen to a desired treatment location, expanding the stent at the treatment location, and removing the catheter from the lumen.

In the case of a balloon expandable stent, the stent is mounted about a balloon disposed on the catheter. Mounting the stent typically involves compressing or crimping the stent onto the balloon. The stent is then expanded by inflating the balloon. The balloon may then be deflated and the catheter withdrawn. In the case of a self-expanding stent, the stent may be secured to the catheter via a constraining member such as a retractable sheath or a sock. When the stent is in a desired bodily location, the sheath may be withdrawn which allows the stent to self-expand.

The stent must be able to satisfy a number of mechanical requirements. First, the stent must be capable of withstanding the structural loads, namely radial compressive forces, imposed on the stent as it supports the walls of a vessel. Therefore, a stent must possess adequate radial strength. Radial strength, which is the ability of a stent to resist radial compressive forces, is due to strength and rigidity around a circumferential direction of the stent. Radial strength and rigidity, therefore, may also be described as, hoop or circumferential strength and rigidity.

Once expanded, the stent must adequately maintain its size and shape throughout its service life despite the various forces that may come to bear on it, including the cyclic loading induced by the beating heart. For example, a radially directed force may tend to cause a stent to recoil inward. Generally, it is desirable to minimize recoil. In addition, the stent must possess sufficient flexibility to allow for crimping, expansion, and cyclic loading. Longitudinal flexibility is important to allow the stent to be maneuvered through a tortuous vascular path and to enable it to conform to a deployment site that may not be linear or may be subject to flexure. Finally, the stent must be biocompatible so as not to trigger any adverse vascular responses.

The structure of a stent is typically composed of scaffolding that includes a pattern or network of interconnecting structural elements often referred to in the art as struts or bar arms. The scaffolding can be formed from wires, tubes, or sheets of material rolled into a cylindrical shape. The scaffolding is designed so that the stent can be radially compressed (to allow crimping) and radially expanded (to allow deployment). A conventional stent is allowed to expand and contract through movement of individual structural elements of a pattern with respect to each other.

Furthermore, it may be desirable for a stent to be biodegradable. In many treatment applications, the presence of a stent in a body may be necessary for a limited period of time until its intended function of, for example, maintaining vascular patency and/or drug delivery is accomplished. Therefore, stents fabricated from biodegradable, bioabsorbable, and/or bioerodable materials such as bioabsorbable polymers should be configured to completely erode only after the clinical need for them has ended.

Additionally, a medicated stent may be fabricated by coating the surface of either a metallic or polymeric scaffolding with a polymeric carrier that includes an active or bioactive agent or drug. Polymeric scaffolding may also serve as a carrier of an active agent or drug. Potential problems with therapeutic coatings for polymeric implantable medical devices, such as stents, include insufficient toughness, slow degradation rate, and poor adhesion.

### SUMMARY OF THE INVENTION

Certain embodiments of the present invention include an implantable medical device comprising a coating above a polymer surface of the device, the coating comprising: an elastomeric copolymer including elastic units and anchor units, the elastic units providing elastomeric properties to the copolymer at physiological conditions, wherein the anchor units enhance adhesion of the coating with the surface polymer.

Further embodiments of the present invention include an implantable medical device comprising a coating above a polymer surface of the device, the coating comprising: an elastomeric copolymer including elastic units and anchor units, the elastic units providing elastomeric properties to the copolymer at physiological conditions, wherein the anchor units enhance adhesion of the coating with the surface polymer, wherein the elastic units are selected from the group consisting of CL, TMC, HB, and DO, wherein the anchor units are selected from the group consisting of LLA and GA, and wherein the surface polymer is selected from the group consisting of PLLA and LPLG.

### BRIEF DESCRIPTION OF THE DRAWINGS

FIG. 1 depicts a view of a stent.

FIG. 2A depicts a cross-section of a stent surface with an elastomeric copolymer coating layer over a substrate.

FIG. 2B depicts a cross-section of a stent surface with an elastomeric copolymer coating layer over a polymeric layer disposed over a substrate of the stent.

FIG. 3 depicts a cross-section of a stent surface with the elastomeric copolymer coating layer over a substrate of the stent showing an interfacial region.

FIG. 4 depicts a cross-section of a stent showing a coating material layer over a swollen surface polymer layer.

FIG. 5 depicts a polymer surface pretreated with a solvent.

FIG. 6 depicts the cross-section of a stent surface with a drug-polymer layer over an elastomeric copolymer primer layer disposed over a substrate of the stent.

### DETAILED DESCRIPTION OF THE INVENTION

Various embodiments of the present invention include an implantable medical device with a coating having an elastomeric polymer above a polymeric surface of the device. The polymeric surface may be a surface of a polymer coating disposed above a substrate that can be composed of metal, polymer, ceramic, or other suitable material. Alternatively, the polymeric surface may be a surface of a polymeric substrate or body. “Above” a surface is defined as higher than or over a surface measured along an axis normal to the surface, but not necessarily in contact with the surface.

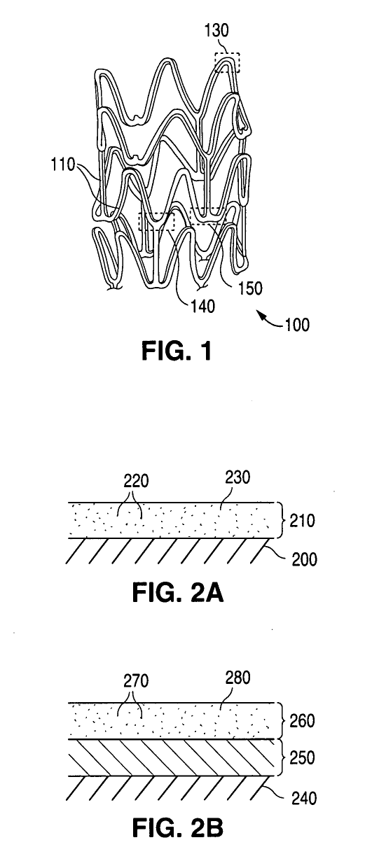

The present invention may be applied to implantable medical devices including, but not limited to, self-expandable stents, balloon-expandable stents, stent-grafts, and grafts (e.g., aortic grafts), and generally expandable tubular devices for various bodily lumen or orifices. A stent can have a scaffolding or a substrate that includes a pattern of a plurality of interconnecting structural elements or struts. FIG. 1 depicts a view of an exemplary stent 100. Stent 100 includes a pattern with a number of interconnecting structural elements or struts 110. In general, a stent pattern is designed so that the stent can be radially compressed (crimped) and radially expanded (to allow deployment). The stresses involved during compression and expansion are generally distributed throughout various structural elements of the stent pattern. The variations in stent patterns are virtually unlimited.

In some embodiments, a stent may be fabricated by laser cutting a pattern on a tube or a sheet rolled into a tube. Representative examples of lasers that may be used include, but are not limited to, excimer, carbon dioxide, and YAG. In other embodiments, chemical etching may be used to form a pattern on a tube.

An implantable medical device can be made partially or completely from a biodegradable, bioabsorbable, biostable polymer, or a combination thereof. A polymer for use in fabricating an implantable medical device can be biostable, bioabsorbable, biodegradable or bioerodable. Biostable refers to polymers that are not biodegradable. The terms biodegradable, bioabsorbable, and bioerodable are used interchangeably and refer to polymers that are capable of being completely degraded and/or eroded when exposed to bodily fluids such as blood and can be gradually resorbed, absorbed, and/or eliminated by the body. The processes of breaking down and absorption of the polymer can be caused by, for example, hydrolysis and metabolic processes.

As indicated above, a medicated implantable medical device, such as a stent, may be fabricated by coating the surface of the device with a drug. For example, a stent can have a coating including a drug dispersed in a polymeric carrier disposed over a substrate of the stent. Such a coating layer may be formed by applying a coating material to a substrate of an implantable medical device, such as a stent. The coating material can be a polymer solution and a drug dispersed in the solution. The coating material may be applied to the stent by immersing the stent in the coating material, by spraying the material onto the stent, or by other methods known in the art. The solvent in the solution is then removed, for example, by evaporation, leaving on the stent surfaces a polymer coating impregnated with the drug.

Stents are typically subjected to stress during use. “Use” includes manufacturing, assembling (e.g., crimping a stent on balloon), delivery of a stent through a bodily lumen to a treatment site, deployment of a stent at a treatment site, and treatment after deployment. Both the underlying scaffolding or substrate and the coating experience stress that result in strain in the substrate and coating. In particular, localized portions of the stent's structure undergo substantial deformation. For example, the apex regions of bending elements 130, 140, and 150 in FIG. 1 experience relatively high stress and strain during crimping, expansion, and after expansion of the stent.

Furthermore, polymer substrates and polymer-based coatings may be particularly vulnerable to mechanical instability during use of a stent. Such mechanical instability for coatings can include fracture and detachment from a substrate, for example, peeling. Some polymers may be susceptible to such mechanical instability due to insufficient toughness at high deformations. Additionally, detachment of coatings may be due to poor adhesion of the polymer-based coating to the substrate or another polymer layer. Therefore, polymer-based coatings are highly susceptible to tearing or fracture, and/or detachment, especially at regions subjected to relatively high stress and strain. Thus, it is important for a polymer-based coating to (1) be tough and have a high resistance to cracking and (2) have good adhesion with an underlying layer or substrate and to have a high resistance to detachment in the range of deformations that occur during crimping, during deployment of a stent, and after deployment.

As indicated above, a device may be composed in whole or in part of materials that degrade, erode, or disintegrate through exposure to physiological conditions within the body until the treatment regimen is completed. The device may be configured to disintegrate and disappear from the region of implantation once treatment is completed. The device may disintegrate by one or more mechanisms including, but not limited to, dissolution and chemical breakdown. The duration of a treatment period depends on the bodily disorder that is being treated. For illustrative purposes only, in treatment of coronary heart disease involving use of stents in diseased vessels, the duration can be in a range from about a month to a few years. However, the duration is typically in a range from about six to twelve months. Thus, it is desirable for polymer-based coatings and substrates of an implantable medical device, such as a stent, to have a degradation time at or near the duration of treatment. Degradation time refers to the time for an implantable medical device to substantially or completely erode away from an implant site.

Embodiments of the present invention can include an elastomeric polymer coating disposed over a polymer surface of a device, such as a stent scaffolding. In certain embodiments, the coating can be disposed directly over the surface of a polymer substrate of a device. FIG. 2A depicts a cross-section of a stent surface with an elastomeric polymer coating layer 210 over a substrate 200. In the embodiment shown in FIG. 2A, elastomeric polymer coating layer 210 includes a drug 220 dispersed in an elastomeric polymer 230. The substrate can be composed of a bioabsorbable polymer.

In other embodiments of the present invention, the elastomeric polymer coating can be over a polymer coating layer that is disposed over a substrate. FIG. 2B depicts a cross-section of a substrate 240 of a stent with a polymeric layer 250 disposed over substrate 240. An elastomeric polymer coating layer 260 is disposed over polymeric layer 250. Coating layer 260 includes a drug 270 dispersed within an elastomeric polymer 280. Polymeric layer 250 can be a primer layer for improving the adhesion of drug-polymer layer 260 to substrate 240. In the embodiment of FIG. 2B, substrate 240 can be metallic, polymeric, ceramic, or other suitable material.

In certain embodiments of the present invention, the elastomeric coating above a polymer surface of an implantable medical device can include an elastomeric copolymer with elastic units and anchor units. In such embodiments, the elastic units provide elastomeric or rubbery properties at physiological conditions to the random copolymer. “Elastic units,” refer to monomer units that form elastic or rubbery polymers at physiological conditions. In addition, the anchor units enhance the adhesion of the random copolymer coating with the surface polymer.

In some embodiments, the elastomeric copolymer can be a random copolymer of elastic units and anchor units. Alternatively, the elastomeric copolymer can be an alternating copolymer with elastic units and anchor units alternating along the polymer chain. In addition, the elastomeric copolymer can include more than one type of elastic unit and more than one type of anchor unit.

In some embodiments, the elastic units, the anchor units, or both can be bioabsorbable. In certain embodiments, all or a majority of the coating may be the elastomeric copolymer. Additionally, the coating can be a therapeutic layer with an active agent or drug mixed or dispersed within the elastomeric copolymer.

As mentioned above, the elastic units coating provides rubbery or elastomeric behavior at physiological conditions to the elastomeric copolymer coating. Such elastomeric properties provide the coating with a high fracture toughness during use of a device such as a stent. An “elastomeric” or “rubbery” polymer refers to a polymer that exhibits elastic deformation through all or most of a range of deformation. Physiological conditions include, but are not limited to, human body temperature, approximately 37° C. Exemplary elastic units can include, but are not limited to, caprolactone (CL), tetramethyl carbonate (TMC), 4-hydroxy butyrate (HB), and dioxanone (DO). In some embodiments, the elastomeric copolymer of the coating can have a glass transition temperature (Tg) below body temperature. Additionally, the elastomeric copolymer may be completely or substantially amorphous.

The anchor units of the elastomeric copolymer are the same as at least one unit in the surface polymer. Additionally, the anchor units can be miscible with the surface polymer. The anchor units can allow portions of segments of the elastomeric copolymer to be miscible with the surface polymer. The degree of adhesion can be increased by increasing the weight percent of the anchor units in the copolymer. In an exemplary embodiment, the surface polymer can be a crystalline or semicrystalline polymer. In such embodiments, the anchor units can be units of such a crystalline or semicrystalline polymer. In an exemplary embodiment, the elastomeric copolymer can have an LLA anchor units and be disposed over a PLLA surface, which can be the surface of a PLLA substrate. In another exemplary embodiment, the elastomeric copolymer can have a LLA, GA, or both LLA and GA anchor units and be disposed over an LPLG surface, which can be the surface of a LPLG substrate.

In additional embodiments, the elastomeric copolymer can include fast degrading units, alternatively or additionally to anchor units, that are selected to increase the degradation rate of the elastomeric copolymer coating. The fast degrading units can be more hydrophilic or more hydrolytically active than the elastic units or the anchor units. Additionally, fast degrading blocks may have acidic and hydrophilic degradation products. The fast degrading units can be glassy at physiological conditions or can be different from units of the surface polymer. In an exemplary embodiment, GA units are fast degrading units in a elastomeric copolymer coating disposed over a PLLA surface polymer.

In some embodiments, the elastomeric copolymer can be a random or alternating copolymer of elastic units and fast degrading units. Alternatively, the copolymer can be a random or alternating copolymer of elastic units, anchor units, and fast degrading units.

Exemplary elastomeric copolymer coatings include PLLA-co-PDO, PLLA-co-PCL, PLLA-co-PTMC, PLLA-co-PDO-co-PTMC, PLLA-co-PGA-co-PDO, PLLA-co-PGA-co-PCL, PLLA-co-PGA-co-PTMC, etc. Such block copolymers may be suitable as coatings over a PLLA or LPLG surface.

Embodiments of the elastomeric polymer coating of the present invention can be applied to a polymer surface so that at least some of the elastomeric polymer is mixed with the surface polymer. In particular, segments of the elastomeric copolymer of the elastomeric copolymer coating that include anchor units can be mixed with the surface polymer. It is believed that an interfacial region between the coating and the surface polymer can form with elastomeric polymer mixed with surface polymer. The anchor units of the elastomeric copolymer can act as a compatibilizer that strengthens the bond between the coating and the coated surface. The interfacial region can enhance the adhesion of the elastomeric polymer coating to the polymer substrate or a polymer surface layer, in general.

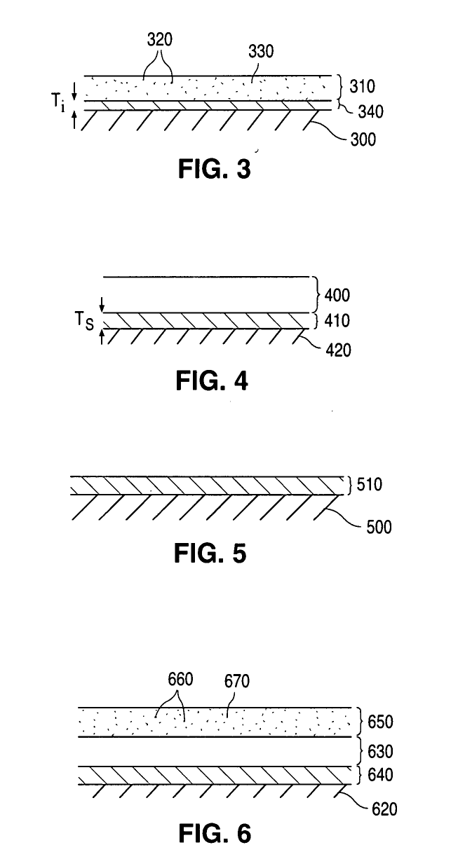

FIG. 3 depicts a cross-section of a stent surface with an elastomeric copolymer coating layer 310 over a substrate 300. Coating layer 310 can be applied to form an interfacial region 340 which can include elastomeric copolymer segments including anchor units mixed with substrate polymer. A drug 320 can be mixed or dispersed within coating layer 310 and interfacial region 340. A thickness Ti of interfacial region 340 can be varied depending on coating application processing parameters.

The enhanced adhesion can allow the use of a tough, high fracture resistant coating that may otherwise have poor adhesion to a polymer substrate of a device. The polymer material for a substrate of a device, such as a stent, may be selected primarily on the basis of strength and stiffness so that the stent substrate can provide support for a lumen. Such substrate polymers can be crystalline or semi-crystalline polymers that are glassy or have a Tg above body temperature. Tough, elastomeric polymers may not necessarily have good adhesion with such a substrate. Embodiments of the elastomeric copolymer disclosed herein allow the use of a tough, high fracture resistant coating over a glassy substrate. Such glassy substrate polymers include PLLA and LPLG.

Embodiments of the elastomeric polymers disclosed herein can be formed by solution-based polymerization. Other methods of forming the elastomeric polymers are also possible, such as, without limitation, melt phase polymerization.

The elastomeric copolymer can be prepared by solution polymerization by preparing a solution including the elastic units, anchor units, optionally fast degrading units, an appropriate solvent, an appropriate initiator, and catalyst. The mixture is allowed to react to form the elastomeric copolymer. The elastomeric copolymer can be removed from the solution through precipitation in a non-solvent of the elastomeric copolymer.

For example, to prepare a PLLA-co-PCL-co-PDO elastomeric copolymer, a solution is formed containing DO units, CL units, and LLA units, a dodecanol initiator, and stannous octoate catalyst in a toluene solvent. The monomers react to form the elastomeric copolymer. The solution can then be added to methanol, which is a non-solvent for the formed elastomeric copolymer, to precipitate the elastomeric copolymer from solution.

In one embodiment, the solvent for use in synthesizing the elastomeric block copolymer is devoid of alcohol functional groups. Such alcohol groups may act as initiators for chain growth in the polymer. Solvents used to synthesize the elastomeric block copolymer include, but are not limited to, chloroform, toluene, xylene, and cyclohexane.

Embodiments of the elastomeric polymer coating of the present invention may be formed over an implantable medical device, such as a stent, by applying a coating material to a polymer surface of the device. The coating material can be a solution including the elastomeric copolymer. The solution can further include an active agent or drug dissolved in a solvent. As discussed above, the coating material may be applied to the stent by immersing the device in the coating material, by spraying the composition onto the device, or by other methods known in the art. The solvent in the applied solution is removed, leaving on the device surfaces the elastomeric polymer coating and optionally drug dispersed within the polymer.

Drying or solvent removal can be performed by allowing the solvent to evaporate at room or ambient temperature. Depending on the volatility of the particular solvent employed, the solvent can evaporate essentially upon contact with the stent. Alternatively, the solvent can be removed by subjecting the coated stent to various drying processes. Drying time can be decreased to increase manufacturing throughput by heating the coated stent. For example, removal of the solvent can be induced by baking the stent in an oven at a mild temperature (e.g., 50° C.) for a suitable duration of time (e.g., 2-4 hours) or by the application of warm air. In an embodiment, a substantial portion of solvent removed may correspond to less than 5%, 3%, or more narrowly, less than 1% of solvent remaining after drying. Depositing a coating of a desired thickness in a single coating stage can result in an undesirably nonuniform surface structure and/or coating defects. Therefore, a coating process can involve multiple repetitions of application, for example, by spraying a plurality of layers.

In some embodiments, the solvent of the coating material is also a solvent for the surface polymer on which the coating material is applied. Specifically, a “solvent” for a given polymer can be defined as a substance capable of dissolving or dispersing the polymer or capable of at least partially dissolving or dispersing the polymer to form a uniformly dispersed mixture at the molecular- or ionic-size level. The solvent should be capable of dissolving at least 0.1 mg of the polymer in 1 ml of the solvent, and more narrowly 0.5 mg in 1 ml at ambient temperature and ambient pressure. The solvent in the coating material can dissolve at least a portion of the surface polymer upon application of the coating material to the polymer surface.

Due to dissolution of a portion of the surface polymer, the coating material near the surface of the surface polymer includes dissolved surface polymer in addition to the elastomeric polymer from the coating material. It is believed that upon removal of the solvent, an interfacial region, as depicted in FIG. 3, is formed that includes segments of the elastomeric copolymer mixed with surface polymer. This interfacial region can be formed due to the miscibility of the surface polymer with the segments including anchor units.

In other embodiments, the solvent in the coating material can be capable of swelling the surface polymer, but is incapable or substantially incapable of dissolving the surface polymer. A solvent that is capable of swelling the surface polymer and is incapable or substantially incapable of dissolving the polymer is understood to mean a sample of the surface polymer swells when immersed in the solvent and the swollen sample of the surface polymer remains in the solvent with a negligible loss of mass for an indefinite period of time at conditions of ambient temperature and pressure.

Solvents for polymers can be found in standard texts (e.g., see Fuchs, in Polymer Handbook, 3rd Edition and Deasy, Microencapsulation and Related Drug Processes, 1984, Marcel Dekker, Inc., New York.) The ability of a polymer to swell and to dissolve in a solvent can be estimated using the Cohesive Energy Density Concept (CED) and related solubility parameter values as discussed by Deasy and can be found in detail in the article by Grulke in Polymer Handbook.

FIG. 4 depicts a cross-section of a stent showing a coating material layer 400 over a swollen surface polymer layer 410. Swollen surface polymer layer 410 is over unswollen polymer coating layer or polymer substrate 420. As indicated above, unswollen surface polymer 420 can either be a substrate of the stent or a polymeric coating over a stent substrate. As shown, swollen surface polymer layer 410 has a thickness Ts. Due to swelling of the surface polymer in swollen surface polymer layer 410, it is believed that segments containing anchor units of the elastomeric polymer in coating material layer 400 penetrate into or mix with the surface polymer in swollen polymer layer 410 prior to removal of the solvent. Upon removal of the solvent, a coating layer is formed over substrate 420.

In some embodiments, a polymeric substrate or polymeric surface coating layer can be pretreated with a solvent that dissolves or swells the surface polymer prior to applying a coating material. FIG. 5 depicts a layer 510 over a substrate or coating layer 500. Layer 510 can be a dissolved layer of surface polymer or a swollen layer of surface polymer. Following pretreatment, the coating material can be applied over the pretreated surface.

In certain embodiments, the coating material solvent is different from the pretreatment solvent. The use of a different solvent for the coating material and the pretreating can provide a degree of flexibility to the coating process. Generally, a treatment with a medicated stent may require a particular drug coating on a coating of a medicated stent. A drug may have an undesirably low or negligible solubility in a selected group of solvents that can dissolve or swell the surface polymer. Thus, a drug coating formed using such a solvent can have an undesirably low concentration of drug. A suitable pretreatment solvent can be used to dissolve or swell the surface polymer and a different solvent can be used as a coating solvent, in which the drug has an acceptable solubility. In general, a required solubility of a drug in a coating solvent is determined by the drug loading required of a particular treatment regimen. Specifically, it is desirable for a drug to have solubility of at least 1 wt % in a solvent for use as a coating material solvent for forming a drug-polymer layer on a stent.

In other embodiments, an elastomeric polymer coating can be a primer layer over a polymer substrate or coating layer. The elastomeric polymer coating can act as a primer layer for a drug-polymer coating layer over the primer layer. The elastomeric polymer primer layer may be formed above a polymeric surface, as described above. The primer coating material can include an elastomeric polymer dissolved in a solvent that can dissolve or swell the surface polymer. A drug-polymer layer can then be formed over the elastomeric polymer primer layer. The drug coating material may include a polymer that is different from the elastomeric polymer and a solvent that is different from the primer coating material solvent. FIG. 6 depicts a drug layer 650 over primer coating layer 630. Drug layer 650 includes a drug 660 mixed or dispersed within a polymer 670. An interfacial layer 640, discussed above, includes segments including anchor units and surface polymer.

In general, representative examples of polymers that may be used to fabricated a substrate of and coatings for an implantable device include, but are not limited to, poly(N-acetylglucosamine) (Chitin), Chitosan, poly(hydroxyvalerate), poly(lactide-co-glycolide), poly(hydroxybutyrate), poly(hydroxybutyrate-co-valerate), polyorthoester, polyanhydride, poly(glycolic acid), poly(glycolide), poly(L-lactic acid), poly(L-lactide), poly(D,L-lactic acid), poly(L-lactide-co-glycolide); poly(D,L-lactide), poly(caprolactone), poly(trimethylene carbonate), polyethylene amide, polyethylene acrylate, poly(glycolic acid-co-trimethylene carbonate), co-poly(ether-esters) (e.g. PEO/PLA), polyphosphazenes, biomolecules (such as fibrin, fibrinogen, cellulose, starch, collagen and hyaluronic acid), polyurethanes, silicones, polyesters, polyolefins, polyisobutylene and ethylene-alphaolefin copolymers, acrylic polymers and copolymers other than polyacrylates, vinyl halide polymers and copolymers (such as polyvinyl chloride), polyvinyl ethers (such as polyvinyl methyl ether), polyvinylidene halides (such as polyvinylidene chloride), polyacrylonitrile, polyvinyl ketones, polyvinyl aromatics (such as polystyrene), polyvinyl esters (such as polyvinyl acetate), acrylonitrile-styrene copolymers, ABS resins, polyamides (such as Nylon 66 and polycaprolactam), polycarbonates, polyoxymethylenes, polyimides, polyethers, polyurethanes, rayon, rayon-triacetate, cellulose, cellulose acetate, cellulose butyrate, cellulose acetate butyrate, cellophane, cellulose nitrate, cellulose propionate, cellulose ethers, and carboxymethyl cellulose.

Additional representative examples of polymers that may be especially well suited for use in embodiments of the present invention include ethylene vinyl alcohol copolymer (commonly known by the generic name EVOH or by the trade name EVAL), poly(butyl methacrylate), poly(vinylidene fluoride-co-hexafluororpropene) (e.g., SOLEF 21508, available from Solvay Solexis PVDF, Thorofare, N.J.), polyvinylidene fluoride (otherwise known as KYNAR, available from ATOFINA Chemicals, Philadelphia, Pa.), ethylene-vinyl acetate copolymers, and polyethylene glycol.

For the purposes of the present invention, the following terms and definitions apply:

For the purposes of the present invention, the following terms and definitions apply:

The “glass transition temperature,” Tg, is the temperature at which the amorphous domains of a polymer change from a brittle vitreous state to a solid deformable, ductile, or rubbery state at atmospheric pressure. In other words, the Tg corresponds to the temperature where the onset of segmental motion in the chains of the polymer occurs. When an amorphous or semicrystalline polymer is exposed to an increasing temperature, the coefficient of expansion and the heat capacity of the polymer both increase as the temperature is raised, indicating increased molecular motion. As the temperature is raised the actual molecular volume in the sample remains constant, and so a higher coefficient of expansion points to an increase in free volume associated with the system and therefore increased freedom for the molecules to move. The increasing heat capacity corresponds to an increase in heat dissipation through movement. Tg of a given polymer can be dependent on the heating rate and can be influenced by the thermal history of the polymer. Furthermore, the chemical structure of the polymer heavily influences the glass transition.

“Stress” refers to force per unit area, as in the force acting through a small area within a plane. Stress can be divided into components, normal and parallel to the plane, called normal stress and shear stress, respectively. True stress denotes the stress where force and area are measured at the same time. Conventional stress, as applied to tension and compression tests, is force divided by the original gauge length.

“Strength” refers to the maximum stress along an axis which a material will withstand prior to fracture. The ultimate strength is calculated from the maximum load applied during the test divided by the original cross-sectional area.

“Strain” refers to the amount of elongation or compression that occurs in a material at a given stress or load.

“Elongation” may be defined as the increase in length in a material which occurs when subjected to stress. It is typically expressed as a percentage of the original length.

“Toughness” is the amount of energy absorbed prior to fracture, or equivalently, the amount of work required to fracture a material. One measure of toughness is the area under a stress-strain curve from zero strain to the strain at fracture. Thus, a brittle material tends to have a relatively low toughness.

Drugs or therapeutic active agent(s) can include anti-inflammatories, antiproliferatives, and other bioactive agents.

An antiproliferative agent can be a natural proteineous agent such as a cytotoxin or a synthetic molecule. Preferably, the active agents include antiproliferative substances such as actinomycin D, or derivatives and analogs thereof (manufactured by Sigma-Aldrich 1001 West Saint Paul Avenue, Milwaukee, Wis. 53233; or COSMEGEN available from Merck) (synonyms of actinomycin D include dactinomycin, actinomycin IV, actinomycin I₁, actinomycin X₁, and actinomycin C₁), all taxoids such as taxols, docetaxel, and paclitaxel, paclitaxel derivatives, all olimus drugs such as macrolide antibiotics, rapamycin, everolimus, structural derivatives and functional analogues of rapamycin, structural derivatives and functional analogues of everolimus, FKBP-12 mediated mTOR inhibitors, biolimus, perfenidone, prodrugs thereof, co-drugs thereof, and combinations thereof. Representative rapamycin derivatives include 40-O-(3-hydroxy)propyl-rapamycin, 40-O-[2-(2-hydroxy)ethoxy]ethyl-rapamycin, or 40-O-tetrazole-rapamycin, 40-epi-(N1-tetrazolyl)-rapamycin (ABT-578 manufactured by Abbott Laboratories, Abbott Park, Ill.), prodrugs thereof, co-drugs thereof, and combinations thereof. In one embodiment, the anti-proliferative agent is everolimus.

An anti-inflammatory drug can be a steroidal anti-inflammatory agent, a nonsteroidal anti-inflammatory agent, or a combination thereof. In some embodiments, anti-inflammatory drugs include, but are not limited to, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecorlimus, prodrugs thereof, co-drugs thereof, and combinations thereof. In one embodiment, the anti-inflammatory agent is clobetasol.

Alternatively, the anti-inflammatory may be a biological inhibitor of proinflammatory signaling molecules. Anti-inflammatory biological agents include antibodies to such biological inflammatory signaling molecules.

In addition, drugs or active can be other than antiproliferative agents or anti-inflammatory agents. These active agents can be any agent which is a therapeutic, prophylactic, or a diagnostic agent. In some embodiments, such agents may be used in combination with antiproliferative or anti-inflammatory agents. These agents can also have anti-proliferative and/or anti-inflammmatory properties or can have other properties such as antineoplastic, antiplatelet, anti-coagulant, anti-fibrin, antithrombonic, antimitotic, antibiotic, antiallergic, antioxidant, and cystostatic agents. Examples of suitable therapeutic and prophylactic agents include synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules which bind to complementary DNA to inhibit transcription, and ribozymes. Some other examples of other bioactive agents include antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides such as antisense oligonucleotides and ribozymes and retroviral vectors for use in gene therapy. Examples of antineoplastics and/or antimitotics include methotrexate, azathioprine, vincristine, vinblastine, fluorouracil, doxorubicin hydrochloride (e.g. Adriamycin® from Pharmacia & Upjohn, Peapack N.J.), and mitomycin (e.g. Mutamycin® from Bristol-Myers Squibb Co., Stamford, Conn.). Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, thrombin inhibitors such as Angiomax a (Biogen, Inc., Cambridge, Mass.), calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacor® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), nitric oxide or nitric oxide donors, super oxide dismutases, super oxide dismutase mimetic, 4-amino-2,2,6,6-tetramethylpiperidine-1-oxyl (4-amino-TEMPO), estradiol, anticancer agents, dietary supplements such as various vitamins, and a combination thereof. Examples of such cytostatic substance include angiopeptin, angiotensin converting enzyme inhibitors such as captopril (e.g. Capoten® and Capozide® from Bristol-Myers Squibb Co., Stamford, Conn.), cilazapril or lisinopril (e.g. Prinivil® and Prinzide® from Merck & Co., Inc., Whitehouse Station, N.J.). An example of an antiallergic agent is permirolast potassium. Other therapeutic substances or agents which may be appropriate include alpha-interferon, and genetically engineered epithelial cells. The foregoing substances are listed by way of example and are not meant to be limiting.

Other bioactive agents may include antiinfectives such as antiviral agents; analgesics and analgesic combinations; anorexics; antihelmintics; antiarthritics, antiasthmatic agents; anticonvulsants; antidepressants; antidiuretic agents; antidiarrheals; antihistamines; antimigrain preparations; antinauseants; antiparkinsonism drugs; antipruritics; antipsychotics; antipyretics; antispasmodics; anticholinergics; sympathomimetics; xanthine derivatives; cardiovascular preparations including calcium channel blockers and beta-blockers such as pindolol and antiarrhythmics; antihypertensives; diuretics; vasodilators including general coronary; peripheral and cerebral; central nervous system stimulants; cough and cold preparations, including decongestants; hypnotics; immunosuppressives; muscle relaxants; parasympatholytics; psychostimulants; sedatives; tranquilizers; naturally derived or genetically engineered lipoproteins; and restenoic reducing agents. Other active agents which are currently available or that may be developed in the future are equally applicable.

### EXAMPLES

The examples set forth below are for illustrative purposes only and are in no way meant to limit the invention. The following examples are given to aid in understanding the invention, but it is to be understood that the invention is not limited to the particular materials or procedures of examples.

### Example 1

### PLLA-co-PDO-co-PCL Random Copolymer Synthesis

In this example, 10 g dioxanone (DO), 10 g caprolactone (CL), and 10 g L-lactide (LLA) as monomers, 0.084 ml stannous octoate as catalyst, 0.22 ml dodecanol as initiator are used.
Step 1: One 500 three neck glassware reactor with a mechanical stirring rod is placed in a glove box which is filled with high purity nitrogen. The reactor is preheated to remove all moisture.
Step 2: DO, CL, LLA, initiator and catalyst are added into the reactor. The mixture is stirred at 110° C. for 40 hours.
Step 3: 200 ml CHCl₃ is then added into reactor to dissolve final product. Finally, the product solution is precipitated into 800 ml methanol, filtered out and dried in vacuum at 80° C. until constant weight.

### Example 2

### Preparation of Coating Solution and Coating Layer on PLLA Stent Backbone

The coating solution is prepared by mixing synthesized copolymer with drug in a solvent. Everolimus, Sirolimus, Paclitaxel, or their derivatives are used as drug, while acetone, dimethylene chloroform, or a mixture thereof is used as solvent. The weight ratio of copolymer to drug is in the range of 1:1 to 5:1, and the weight percent of copolymer in the solution is in the range of 0.1-4 wt %. The coating layer is prepared through spray/dip/drop coating of solution on stent backbone.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

What is claimed is:

1. An implantable medical device comprising a coating above a polymer surface of the device, the coating comprising:
an elastomeric copolymer including elastic units and anchor units, the elastic units providing elastomeric properties to the copolymer at physiological conditions, wherein the anchor units enhance adhesion of the coating with the surface polymer.

2. The device of claim 1, wherein the device is a stent.

3. The device of claim 1, wherein the copolymer is a random copolymer or an alternating copolymer.

4. The device of claim 1, wherein the anchor units are the same as at least some units of the surface polymer.

5. The device of claim 1, wherein the elastomeric copolymer further comprises fast degrading units that increase the degradation rate of the coating, the fast degrading units being glassy and different from units of the surface polymer.

6. The device of claim 5, wherein the surface polymer comprises PLLA and the fast-degrading units comprise GA.

7. The device of claim 1, wherein the elastic and anchor units of the copolymer are bioabsorbable.

8. The device of claim 1, wherein a body of the device is formed from a bioabsorbable polymer.

9. The device of claim 1, wherein a majority of the coating comprises the elastomeric copolymer.

10. The device of claim 1, wherein the polymer surface comprises a surface of a substrate of the device or a surface of a coating layer above the substrate of the device.

11. The device of claim 1, wherein the coating comprises an active agent.

12. The device of claim 1, wherein the elastic block, the anchor block, and the surface polymer are biodegradable.

13. The device of claim 1, wherein the surface polymer is PLLA and the anchor units are selected from the group consisting of LLA and GA.

14. The device of claim 1, wherein the elastic units are selected from the group consisting of CL, TMC, HB, and DO.

15. An implantable medical device comprising a coating above a polymer surface of the device, the coating comprising:
an elastomeric copolymer including elastic units and anchor units, the elastic units providing elastomeric properties to the copolymer at physiological conditions, wherein the anchor units enhance adhesion of the coating with the surface polymer, wherein the elastic units are selected from the group consisting of CL, TMC, HB, and DO, wherein the anchor units are selected from the group consisting of LLA and GA, and wherein the surface polymer is selected from the group consisting of PLLA and LPLG.

16. The device of claim 15, wherein the device is a stent.

17. The device of claim 15, wherein the copolymer is a random copolymer or an alternating copolymer.

18. The device of claim 15, wherein the elastomeric copolymer further comprises GA units that increase the degradation rate of the coating.

19. The device of claim 15, wherein the polymer surface is a surface of the body of the device, the body of the device being formed from the surface polymer.
